(19) United States
(12) Patent Application Publication
(10) Pub. No.: US 2025/0009783 A1
(43) Pub. Date: Jan. 9, 2025

(54) SULFONATED HYALURONIC ACID COMPOUND, PREPARATION METHOD THEREFOR AND APPLICATION THEREOF

(71) Applicant: UNIVERSITY OF MACAU, Macau (CN)

(72) Inventors: Chunming WANG, Macau (CN); Jiaxi CHEN, Macau (CN); Daping XIE, Macau (CN); Zhe ZHANG, Macau (CN)

(21) Appl. No.: 18/702,339

(22) PCT Filed: Nov. 16, 2021
(86) PCT No.: PCT/CN2021/130827

§ 371 Date: Apr. 18, 2024

(30) Foreign Application Priority Data
Oct. 19, 2021 (CN) 202111213457.7

(51) Int. Cl. A61K 31/728; C08B 37/08

(52) U.S. Cl. CPC A61K 31/728; C08B 37/0072

(57) ABSTRACT

The present invention relates to the cross-technical fields of medicine, material chemistry, glycobiology and the like, in particular to a sulfonated hyaluronic acid compound, a preparation method therefor and an application thereof. The structural formula of the sulfonated hyaluronic acid compound is shown as in formula (I), wherein R is alkali metal cation or hydrogen; R₁, R₂, R₃ and R₄ are respectively independently selected from hydrogen or sulfonate ions and R₁, R₂, R₃ and R₄ cannot be hydrogen at the same time, 10<n<4000 and n is an integer. The sulfonated hyaluronic acid compound and the LTBP protein have stronger interaction force, so that a combination of the LTBP and ECM is prevented, so that the mechanical force is insufficient, TGF-β cannot be activated, and fibrosis is inhibited from the source of signal transduction.

### CROSS REFERENCE TO RELATED APPLICATIONS

The present disclosure claims priority to Chinese Patent Application No. 202111213457.7, filed with the China Patent Office on Oct. 19, 2021 and entitled “SULFONATED HYALURONIC ACID COMPOUND, PREPARATION METHOD THEREOF, AND APPLICATIONS THEREOF”, which is incorporated herein by reference in its entirety.

### TECHNICAL FIELD

The present disclosure relates to an interdisciplinary technical field combining medicine, material chemistry, glycobiology and the like, and in particular to a sulfonated hyaluronic acid compound, a preparation method therefor, and the use thereof.

### BACKGROUND ART

Fibrosis is the main cause of disability and death resulting from many diseases, and can occur in many organs. Specifically, fibrosis is associated with many diseases such as liver cirrhosis, hepatitis, non-alcoholic steatohepatitis, chronic kidney disease, myocardial infarction, heart failure, idiopathic pulmonary fibrosis, diabetes and scleroderma, which poses a serious threat to human health and life.

As of 2020, there were no drugs approved by the regulatory agency that can avoid or reverse the fibrosis process. Currently, drugs that target the metabolic process and partially inhibit or alleviate fibrosis are mainly used in clinical practice. Among them, most anti-fibrotic drugs, mainly small-molecule drugs, have anti-fibrotic, anti-inflammatory and anti-oxidant effects, and can delay viscera function decline and disease progression caused by fibrosis. However, their specific pharmacological basis is unclear and their effects are relatively poor. There are also some drugs that mainly target proteins and receptors downstream of the TGF-β-smad pathway. Their pharmacological mechanisms of action are clear, but their clinical use can only ameliorate fibrosis or is only effective in the early stage, and has certain adverse reactions. Current researches focus on the development of small-molecule drugs and the inhibition of fibrosis downstream of signalling pathways.

During the occurrence and development of fibrosis, transforming growth factor-β (TGF-β) has functions of promoting collagen gene expression, promoting the synthesis and deposition of extracellular matrix (ECM), etc., thus being one of the most important initiation factors of fibrosis. TGF-β can regulate the physiological process and play a role through the TGF-β-smad signalling pathway. Currently, most researches focus on the regulation and control of downstream signals of TGF-β. However, there are few studies on the regulation and control of TGF-β activation process and the inhibition at the source of the signalling pathway, so as to achieve an anti-fibrotic effect, and there are no drugs that can perform inhibition at the source of the signalling pathway, so as to achieve anti-fibrosis.

### SUMMARY OF THE INVENTION

The present disclosure provides a sulfonated hyaluronic acid compound of a structural formula of:

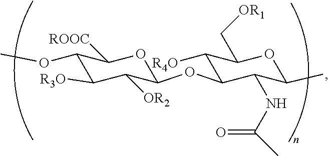

wherein R is an alkali metal cation or hydrogen; R₁, R₂, R₃ and R₄ are each independently selected from hydrogen or a sulfonate ion, and R₁, R₂, R₃ and R₄ cannot be hydrogen simultaneously; and n is an integer of 10<n<4000.

In some embodiments, R is a sodium ion or a potassium ion or hydrogen; and R₁, R₂, R₃ and R₄ are each independently selected from hydrogen or a sulfonate ion, and R₁, R₂, R₃ and R₄ cannot be hydrogen simultaneously.

In some embodiments, R is an alkali metal cation or hydrogen; R₁, R₂, R₃ and R₄ are a sulfonate ion; and n is an integer of 2100<n<4000.

The present disclosure also provides a preparation method of the above-mentioned sulfonated hyaluronic acid compound, performed according to the following synthetic route:

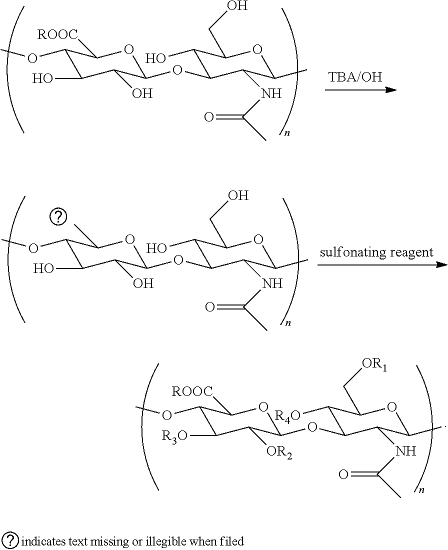

In some embodiments, the molecular weight of the raw hyaluronic acid used in the preparation method is 1500 kDa or less; for example, the molecular weight of the raw hyaluronic acid is any numerical value of <10 kDa or in a range of 100-200 kDa and in a range of 800 kDa-1500 kDa; optionally, the molecular weight of the raw hyaluronic acid is 800 kDa-1500 kDa.

In some embodiments, the sulfonating reagent used in the preparation method is pyridine sulphur trioxide.

In some embodiments, the preparation of the above-mentioned sulfonated hyaluronic acid compound comprises the steps of dissolving the raw hyaluronic acid and then mixing with TBAOH for reaction; followed by lyophilization to form a hyaluronic acid intermediate powder; then mixing the hyaluronic acid intermediate powder with a sulfonating reagent, and adjusting the pH of reaction system to 8-9; followed by dialysis.

The present disclosure provides hyaluronic acid nanoparticles with a structural formula of:

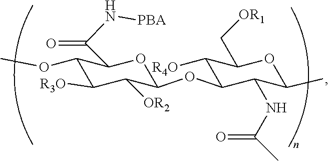

wherein R₁, R₂, R₃ and R₄ are a sulfonate ion; and n is an integer of 10<n<4000; optionally, 2100<n<4000.

The present disclosure also provides a preparation method for the above-mentioned hyaluronic acid nanoparticles, by the following synthetic route:

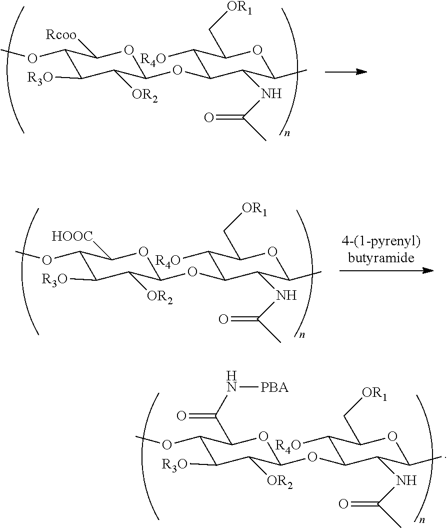

In some embodiments, the preparation method comprises subjecting the above-mentioned sulfonated hyaluronic acid compound to ion exchange to form a sulfonated hyaluronic acid intermediate; and then mixing with an activator; followed by mixing with 4-(1-pyrenyl) butyramide and dialysis.

In some embodiments, the activator is carbodiimide and N-hydroxysuccinimide.

The present disclosure also provides a glycobiological material comprising the above-mentioned sulfonated hyaluronic acid compound or hyaluronic acid nanoparticles.

The present disclosure provides the use of the above-mentioned sulfonated hyaluronic acid compound or hyaluronic acid nanoparticles or glycobiological material in inhibiting fibrosis or inhibiting the activation of TGF-β.

The present disclosure also provides a method for inhibiting fibrosis or inhibiting the activation of TGF-β in a subject in need thereof, comprising administering to the subject the above-mentioned sulfonated hyaluronic acid compound or hyaluronic acid nanoparticles or glycobiological material.

In some embodiments, the fibrosis includes tissue fibrosis; optionally, the fibrosis includes pulmonary fibrosis, hepatic fibrosis, cardiac fibrosis, pancreatic fibrosis and renal fibrosis.

### BRIEF DESCRIPTION OF THE DRAWINGS

In order to more clearly illustrate the technical solutions of the examples of the present disclosure, the drawings used in the examples will be briefly introduced below. It should be understood that the following drawings only show certain examples of the present disclosure, and therefore should not be considered as limiting the scope. For a person skilled in the art, other related drawings also can be obtained according to the drawings without creative efforts.

FIG. 1 is a diagram showing the characterization results of the sulfonated hyaluronic acid compounds provided in examples 1-9 of the present disclosure.

FIG. 2 is a diagram showing the NMR characterization results of the 4-(1-pyrenyl) butyramide provided in example 10 of the present disclosure.

FIG. 3 is a diagram showing the NMR characterization results of the hyaluronic acid nanoparticles provided in examples 10-12 of the present disclosure.

FIG. 4 is a diagram showing the characterization of the particle size and morphology of the hyaluronic acid nanoparticles provided in examples 10-12 of the present disclosure.

FIG. 5 is a test diagram provided for test example 1 of the present disclosure.

FIG. 6 is a test diagram provided for test example 2 of the present disclosure.

### DETAILED DESCRIPTION OF EMBODIMENTS

A detailed description of the embodiments of the present disclosure will be given below with reference to the accompanying drawings and examples, but those skilled in the art will understand that the following examples are only used to illustrate the present disclosure and should not be regarded as limiting the scope of the present disclosure. If specific conditions are not specified in the examples, conventional conditions or conditions recommended by a manufacturer are followed. The reagents or instruments used therein for which manufacturers are not specified are all conventional products that are commercially available.

To function, TGF-β needs to be in the form of active TGF-β. The newly synthesized TGF-β forms a small dormant complex without activity with the latency-associated peptide (LAP) in the form of a non-covalent bond, and the LAP then forms a large latent complex (LLC) with the latent TGF-β binding protein (LTBP) via a disulfide bond. The activation of TGF-β requires the interaction of the LTBP with an extracellular matrix (ECM) and the generation of a certain strength of mechanical force, so that the removal of the “closely-attached” LAP from the TGF-β is promoted by traction, and TGF-β becomes active. Therefore, the inventors have studied that by modulating the mechanical force during TGF activation to be insufficient and thus inhibiting its activation, the formation of active TGF-β can be reduced, thereby inhibiting fibrosis at the root.

Given this, the present disclosure provides a sulfonated hyaluronic acid compound with the following structural formula:

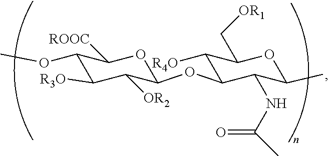

wherein R is an alkali metal cation or hydrogen; R₁, R₂, R₃ and R₄ are each independently selected from hydrogen or a sulfonate ion, and R₁, R₂, R₃ and R₄ cannot be hydrogen simultaneously; and n is an integer of 10<n<4000.

The sulfonated hyaluronic acid provided by the present disclosure has a high affinity with LTBP, LTBP can bind to ECM under normal physiological conditions to provide sufficient mechanical force to promote TGF-β activation, while the sulfonated hyaluronic acid can have a higher affinity with LTBP to prevent LTBP from binding to ECM, so that the mechanical force is insufficient to activate TGF-β, and fibrosis is inhibited at the source of signalling, and therefore the sulfonated hyaluronic acid can be use for ameliorating or treating fibrosis in subjects in need.

In some embodiments, R is a sodium ion or a potassium ion or hydrogen; and R₁, R₂, R₃ and R₄ are each independently selected from hydrogen or a sulfonate ion, and R₁, R₂, R₃ and R₄ cannot be hydrogen simultaneously.

The present disclosure also provides a preparation method for the sulfonated hyaluronic acid compound described in the above-mentioned embodiments, and the sulfonated hyaluronic acid compound is synthesized with reference to the following synthetic route:

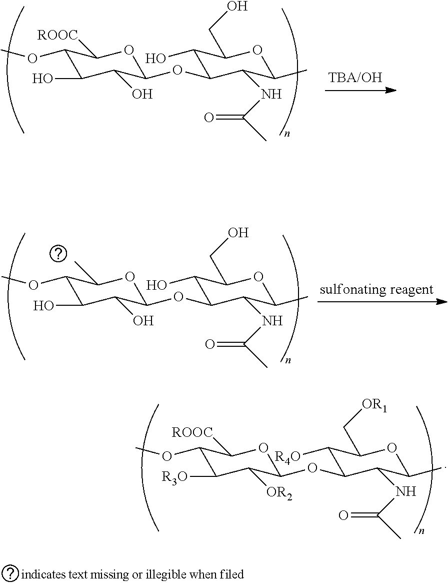

In some embodiments, the raw hyaluronic acid is dissolved and then mixed with tetrabutylammonium hydroxide (TBAOH) for reaction; followed by lyophilization to form a hyaluronic acid intermediate powder; then the hyaluronic acid intermediate powder is mixed with a sulfonating reagent, and the pH of reaction system is adjusted to 8-9; followed by dialysis. For the raw hyaluronic acid, a hyaluronic acid with any molecular weight can be used. For example, the molecular weight of the raw hyaluronic acid is 1500 kDa or less; optionally, the molecular weight of the raw hyaluronic acid is any numerical value of <10 kDa or in a range of 100-200 kDa and in a range of 800 kDa-1500 kDa. For the sulfonating reagent to be used, an existing sulfonating reagent can also be used. For example, the sulfonating reagent is selected from pyridine sulphur trioxide.

Additionally, the present disclosure provides hyaluronic acid nanoparticles of a structural formula of:

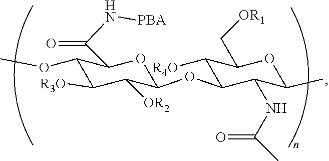

wherein R₁, R₂, R₃ and R₄ are a sulfonate ion; and n is an integer of 10<n<4000. In some embodiments, the covalent binding of 4-(1-pyrenyl) butyramide with sulfonated hyaluronic acid is used by the present disclosure to form hydrophilic and hydrophobic ends, which can be self-assembled to form whisk-like nanoparticles surrounded by hyaluronic acid chains, and different nanoparticle sizes and compactness can be adjusted by adjusting the amount of 4-(1-pyrenyl) butyramide. Hyaluronic acid polysaccharide chains can enrich the LLCs in the tissue and prevent them from contacting with ECM and activating TGF-β, which play a role in inhibiting TGF-β activation.

The present disclosure also provides a preparation method for the hyaluronic acid nanoparticles described in the above-mentioned embodiments, and the hyaluronic acid nanoparticles are synthesized with reference to the following synthetic route:

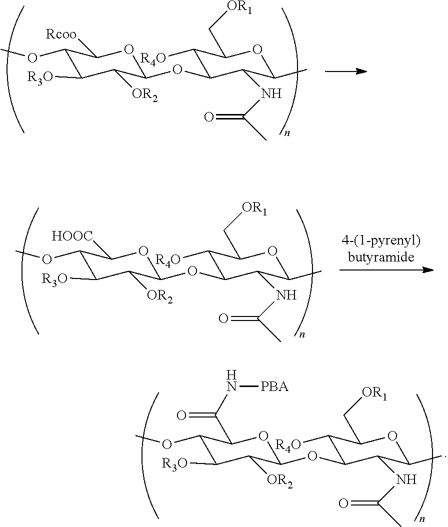

In some embodiments, the sulfonated hyaluronic acid compound is subjected to ion exchange to form a sulfonated hyaluronic acid intermediate, and then mixed with an activator, and next with 4-(1-pyrenyl) butyramide, followed by dialysis. Optionally, the activator is carbodiimide and N-hydroxysuccinimide.

Additionally, the present disclosure provides a glycobiological material comprising the sulfonated hyaluronic acid compound or hyaluronic acid nanoparticles described in the above-mentioned embodiments.

The present disclosure also provides the use of the sulfonated hyaluronic acid compound described in the above-mentioned embodiments or the hyaluronic acid nanoparticles described in the above-mentioned embodiments or the glycobiological material described in the above-mentioned embodiments in the inhibition of fibrosis; wherein the fibrosis includes tissue fibrosis; for example, the fibrosis includes pulmonary fibrosis, hepatic fibrosis, cardiac fibrosis, pancreatic fibrosis and renal fibrosis; and the drugs are drugs for inhibiting TGF-β activation.

### EXAMPLES

The characteristics and performance of the present disclosure will be further described in detail below in combination with the examples.

### Example 1

This example provided a sulfonated hyaluronic acid compound and a preparation method therefor.

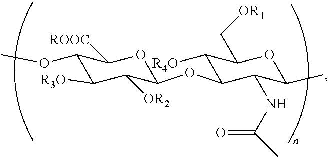

wherein R₁═SO₃⁻, R₂═H, R₃═H, and R₄═H; and 10<n<30.

The preparation method comprises:

3 g of low-molecular-weight hyaluronic acid (with a molecular weight<10 kDa) was dissolved in 300 ml of deionized water, and stirred with 6 ml of 25% tetrabutylammonium hydroxide for reaction at room temperature for 2 h, followed by lyophilization to obtain a hyaluronic acid intermediate powder soluble in organic reagents. 300 mg of hyaluronic acid intermediate powder was taken and dissolved in anhydrous dimethylformamide, and stirred for dispersion. Pyridine sulphur trioxide with a low degree of substitution (0.9 g) was taken and dissolved in dimethylformamide, and then hyaluronic acid solution was added under an ice bath for 1 h of reaction. Then, water was added to terminate the reaction, and then pH was adjusted to 8.5 by sodium hydroxide, followed by dialysis in water for 2 days and lyophilization to obtain the sulfonated hyaluronic acid compound, designated as S-HA-1.

It should be noted that sulfonated hyaluronic acids with a low degree of substitution: R₁═SO₃⁻, R₂═H, R₃═H, and R₄═H; Sulfonated hyaluronic acids with a medium degree of substitution: R₁═SO₃⁻, R₂═SO₃⁻, R₃═H, and R₄═H; Sulfonated hyaluronic acids with a high degree of substitution: R₁═SO₃⁻, R₂═SO₃⁻, R₃═SO₃⁻, and R₄═SO₃⁻.

Hyaluronic acid with a low molecular weight: (<10 kDa); and 10<n<30; Hyaluronic acid with a medium molecular weight: (100 kDa-200 kDa); and 260<n<530; Hyaluronic acid with a high molecular weight: (800 kDa-1500 kDa); and 2100<n<4000.

### Example 2

This example provided a sulfonated hyaluronic acid compound and a preparation method therefor.

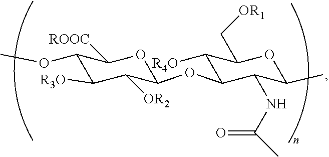

wherein R₁═SO₃⁻, R₂═SO₃⁻, R₃═H, and R₄═H; and 10<n<30.

The preparation method was basically the same as that provided in example 1, except that pyridine sulphur trioxide had a medium degree of substitution (2.28 g), the raw hyaluronic acid was a hyaluronic acid with a low molecular weight, and the resulting sulfonated hyaluronic acid compound was designated as S-HA-2.

### Example 3

This example provided a sulfonated hyaluronic acid compound and a preparation method therefor.

The sulfonated hyaluronic acid compound has a structural formula of:

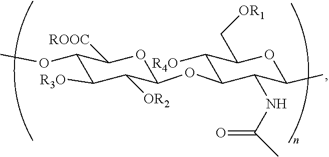

wherein R₁═SO₃⁻, R₂═SO₃⁻, R₃═SO₃⁻ and R₄═SO₃⁻; and 10<n<30.

The preparation method was basically the same as that provided in example 1, except that pyridine sulphur trioxide had a high degree of substitution (3.66 g), the raw hyaluronic acid was a hyaluronic acid with a low molecular weight, and the resulting sulfonated hyaluronic acid compound was designated as S-HA-3.

### Example 4

This example provided a sulfonated hyaluronic acid compound and a preparation method therefor.

The sulfonated hyaluronic acid compound has a structural formula of:

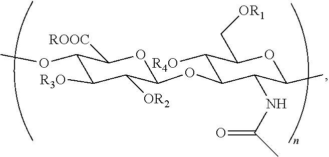

wherein R₁═SO₃⁻, R₂═H, R₃═H, and R₄═H; and 260<n<530.

The preparation method was basically the same as that provided in example 1, except that the molecular weight of the hyaluronic acid used was 100 kDa-200 kDa, the pyridine sulphur trioxide had a low degree of substitution, and the resulting sulfonated hyaluronic acid compound was designated as S-HA-4.

### Example 5

This example provided a sulfonated hyaluronic acid compound and a preparation method therefor.

The sulfonated hyaluronic acid compound has a structural formula of:

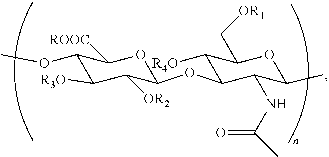

wherein R₁═SO₃⁻, R₂═SO₃⁻, R₃═H, and R₄═H; and 260<n<530.

The preparation method was basically the same as that provided in example 1, except that the molecular weight of the hyaluronic acid used was 100 kDa˜200 kDa, and pyridine sulphur trioxide had a medium degree of substitution (2.28 g). The resulting sulfonated hyaluronic acid compound was designated as S-HA-5.

### Example 6

This example provided a sulfonated hyaluronic acid compound and a preparation method therefor.

The sulfonated hyaluronic acid compound has a structural formula of:

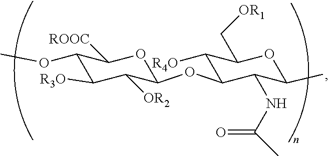

wherein R₁═SO₃⁻, R₂═SO₃⁻, R₃═SO₃⁻, and R₄═SO₃⁻; and 260<n<530.

The preparation method was basically the same as that provided in example 1, except that the molecular weight of the hyaluronic acid used was 100 kDa-200 kDa, the pyridine sulphur trioxide had a high degree of substitution (3.66 g), and the resulting sulfonated hyaluronic acid compound was designated as S-HA-6.

### Example 7

This example provided a sulfonated hyaluronic acid compound and a preparation method therefor.

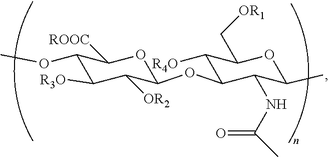

wherein R₁═SO₃⁻, R₂═H, R₃═H, and R₄═H; and 2100<n<4000.

The preparation method was basically the same as that provided in example 1, except that the molecular weight of hyaluronic acid used was a high molecular weight of 800 kDa-1500 kDa, the pyridine sulphur trioxide had a low degree of substitution, and the resulting sulfonated hyaluronic acid compound was designated as S-HA-7.

### Example 8

This example provided a sulfonated hyaluronic acid compound and a preparation method therefor.

The sulfonated hyaluronic acid compound has a structural formula of:

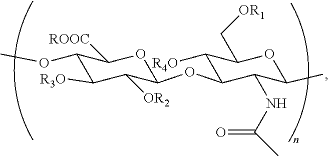

wherein R₁═SO₃⁻, R₂═SO₃⁻, R₃═H, and R₄═H; and 2100<n<4000.

The preparation method was basically the same as that provided in example 1, except that the molecular weight of the hyaluronic acid used was a high molecular weight of 800 kDa-1500 kDa, the pyridine sulphur trioxide had a medium degree of substitution, and the resulting sulfonated hyaluronic acid compound was designated as S-HA-8.

### Example 9

This example provided a sulfonated hyaluronic acid compound and a preparation method therefor.

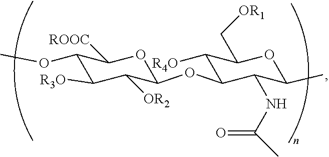

wherein R₁═SO₃⁻, R₂═SO₃⁻, R₃═SO₃⁻, and R₄═SO₃⁻; and 2100<n<4000.

The preparation method was basically the same as that provided in example 1, except that the molecular weight of the hyaluronic acid used was a high molecular weight of 800 kDa-1500 kDa, the pyridine sulphur trioxide had a high degree of substitution, and the resulting sulfonated hyaluronic acid compound was designated as S-HA-9.

### Product Characterization

Characterization of the sulfonated hyaluronic acid compounds synthesized in examples 1-9, i.e., S-HA-1 to S-HA-9, was provided.

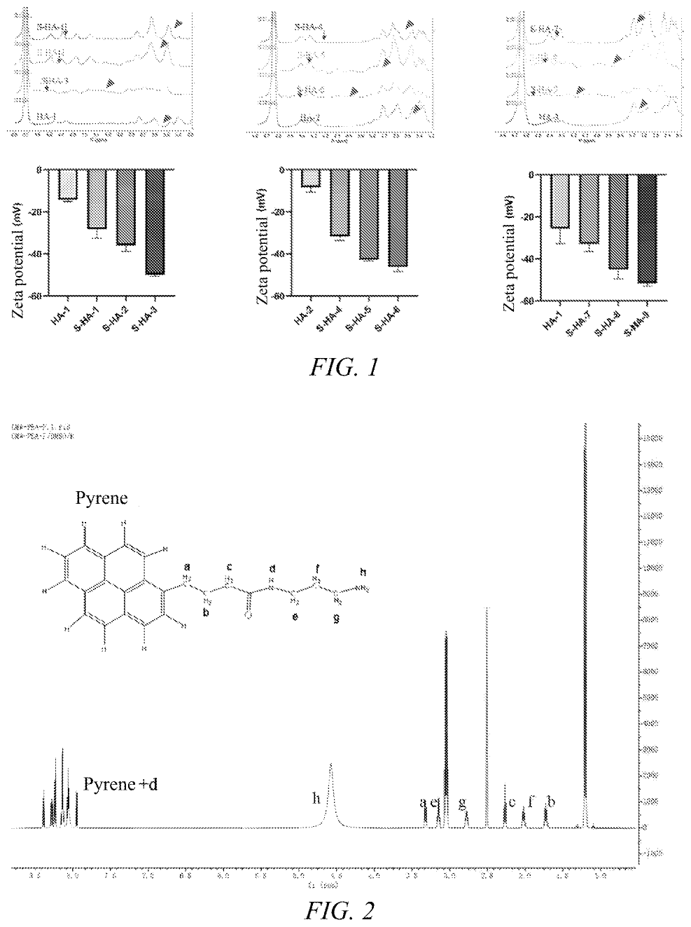

The S-HA-1 to S-HA-9 were characterized using NMR technology. The results were shown in FIG. 1, and the analysis was as follows. The arrow mark represented the peak shift of the methylene proton in C-6, and the triangle represented the peak shift of the adjacent hydroxyl, indicating that the sulfonation substitution degree of S-HA-1, S-HA-2 and S-HA-3 were in an ascending sequence, compared to that of HA-1 with a molecular weight<10 kDa; indicating that the sulfonation substitution degree of S-HA-4, S-HA-5 and S-HA-6 were in an ascending sequence, compared to that of HA-2 with a molecular weight of 100 kDa-200 kDa; and indicating that the sulfonation substitution degree of S-HA-7, S-HA-8 and S-HA-9 were in an ascending sequence, compared to that of HA-3 with a molecular weight of 800 kDa-1500 kDa.

The sulfonation substitution degrees of S-HA-1 to S-HA-9 were characterized using a potentiometer, and the results were shown in FIG. 1. As can be seen from the analysis, a descending sequence of the potentials of the groups indicates an ascending sequence of the sulfonation substitution degree.

### Example 10

This example provided hyaluronic acid nanoparticles and a preparation method therefor.

The hyaluronic acid nanoparticles have a structural formula of.

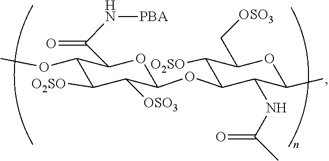

2100<n<4000.

The preparation method comprises:

### S1. Preparation of 4-(1-pyrenyl) butyramide

500 mg (1.73 eq) of pyrenebutyric acid was dissolved in 10 ml of anhydrous dimethylformamide, and then 1970 mg (3 eq) of 1-[bis(dimethylamino)methylene]-1H-1,2,3-triazolo[4,5-b]pyridinium 3-oxide hexafluorophosphate (HATU), 903 mg (5.19 eq) of propylene diamine, and 669 mg (5.19 eq) of diisopropylethylamine (DIPEA) were added, and then stirred for reaction at room temperature for 16 h to obtain an ester with carboxyl connected to ethylene diamine, followed by extraction using ethyl acetate and dichloromethane in a ratio of 1:1 to remove the byproducts and the incompletely reacted raw materials, and a reversed-phase silica gel chromatography column to obtain pure 4-(1-pyrenyl) butyramide.

The 4-(1-pyrenyl) butyramide was characterized using NMR technology. The results were shown in FIG. 2, and the analysis was as follows. The chemical shift 8-8.5 clearly showed nine hydrogens on the benzene ring, and the two hydrogens at the chemical shift 3.5 represent the successful covalent binding of ethylene diamine, indicating the successful synthesis of 4-(1-pyrenyl) butyramide (PBA).

### S2. Synthesis of Hyaluronic Acid Nanoparticles

The sulfonated hyaluronic acid compound prepared in example 9 was passed through an ion exchange resin, and sodium hyaluronate was replaced with sulfonated hyaluronic acid, followed by lyophilization to obtain a sulfonated hyaluronic acid intermediate. 100 mg of the resulting sulfonated hyaluronic acid was dissolved in 10 ml of dimethyl sulfoxide. After the dissolution, 50 mg of carbodiimide and 30 mg of N-hydroxysuccinimide were added. After 30 min of activation, 60 mg of 4-(1-pyrenyl) butyramide was added, and dissolved by ultrasound, and stirred at room temperature for 16 h, followed by dialysis in water for 2 days, and then lyophilization to obtain hyaluronic acid nanoparticles, designated as S-HA-PBA-1.

### Example 11

This example provided hyaluronic acid nanoparticles and a preparation method therefor. The hyaluronic acid nanoparticles were prepared with reference to the preparation method provided in example 10, except that the amount of 4-(1-pyrenyl) butyramide was 50 mg, and the resulting hyaluronic acid nanoparticles were designated as S-HA-PBA-2.

### Example 12

This example provided hyaluronic acid nanoparticles and a preparation method therefor. The hyaluronic acid nanoparticles were prepared with reference to the preparation method provided in example 10, except that the amount of 4-(1-pyrenyl) butyramide was 90 mg, and the resulting hyaluronic acid nanoparticles were designated as S-HA-PBA-3.

### Product Characterization

Characterization of the hyaluronic acid nanoparticles prepared in examples 10-12, i.e., S-HA-PBA-1 to S-HA-PBA-3, was provided.

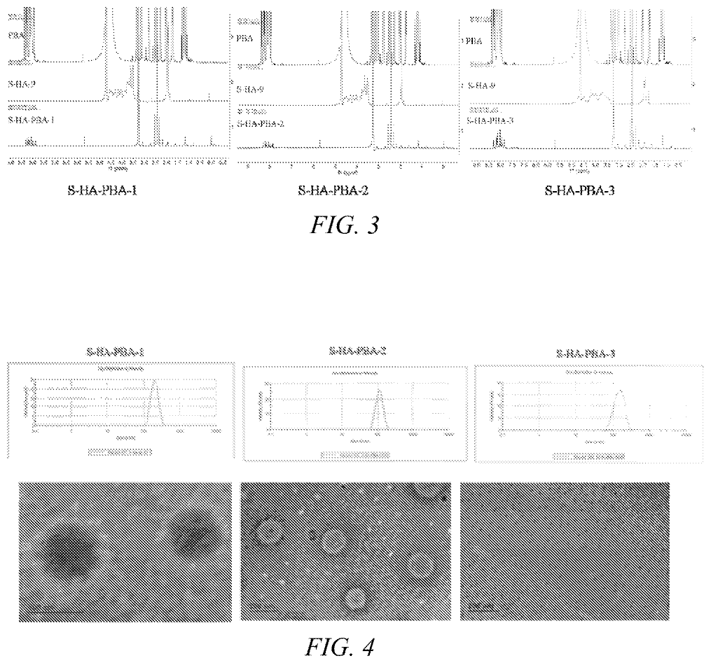

The S-HA-PBA-1 to S-HA-PBA-3 were characterized using NMR technology and infrared spectroscopy technology. The results were shown in FIG. 3, and the analysis was as follows. The chemical shift 8-8.5 ppm illustrated the position of the hydrogen on the benzene ring, proving the successful covalent binding of S-HA-PBA-1, S-HA-PBA-2 and S-HA-PBA-3, with the grafting ratio being calculated from the ratio of the hydrogen on pyrene ring to the hydrogen of amino. The grafting ratio of S-HA-PBA-1 was 17.5%, the grafting ratio of S-HA-PBA-2 was 53.2%, and the grafting ratio of S-HA-PBA-3 was 86.2%.

The particle size and morphology of S-HA-PBA-1 to S-HA-PBA-3 were characterized by a particle size analyser and a transmission electron microscope. The results were shown in FIG. 4, and the analysis was as follows. Both particle size analyser and transmission electron microscope showed that the nanoparticles were successfully synthesized and the particle size was about 100 nm-200 nm.

### Test Example 1

### Affinity Test

Method: Biotinylated LTBP was prepared, and the sulfonated hyaluronic acid compounds of examples 1-9 were respectively used to prepare samples to be tested that had five different concentrations (0.5 mol/L, 1 mol/L, 2 mol/L, 5 mol/L, and 7 mol/L). The binding and dissociation curves were respectively obtained on a bio-layer interferometer by a procedure of buffer 60 s, protein loading 60 s, buffer 60 s, binding 180 s, and dissociation 180 s. The value of dissociation constant KD was calculated according to molecular weight, and the smaller the dissociation constant, the stronger the affinity.

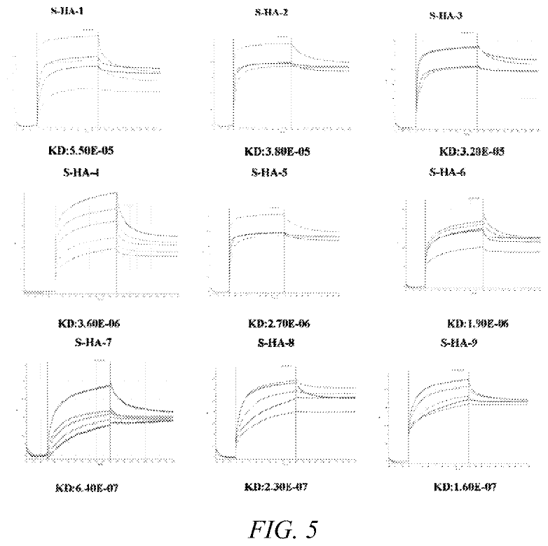

See FIG. 5 for the results. As can be seen from FIG. 5, among the nine sulfonated hyaluronic acid compounds of examples 1-9 with different sulfonation degree, S-HA-9 had the strongest affinity.

### Test Example 2

### Test Using TGF-β Activity Reporter Cell

Method: CAGA-TGF-β activity reporter cells were plated (12-well plate) until the confluence reached 65%. Samples and reagents were added to different wells, respectively. The grouping and concentration were as follows. Group 1:2 ml of TGF-β (50 ng/ml); Group 2:2 ml of pro-TGF-β1 (200 ng/ml); Group 3:2 ml of LLC (200 ng/ml)+S-HA-9 (20 ug/ml); Group 4:2 ml of LLC (200 ng/ml)+PBS (1×); Group 5:2 ml of LLC (200 ng/ml)+S-HA-PBA-1 (20 ug/ml), the cells were stimulated overnight, the samples were collected, and cell lysate was obtained for luciferase test.

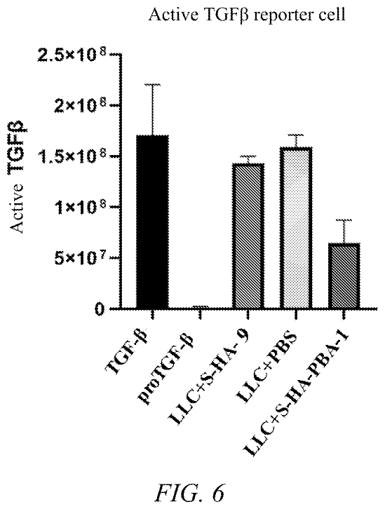

See FIG. 6 for the results. As can be seen from FIG. 6, the sulfonated hyaluronic acid nanoparticles could inhibit the release of TGF-β. As can be seen from the figure, TGF-β stimulated the production of active TGF-β, and proTGF-β stimulated the production of a very small amount of active TGF-β, indicating that the reporter cells were available, which could prove the release behaviour of active TGF-β from the biological experimental level, and the results were credible; the LLC+S-HA-9 group released less active TGF-β than the LLC+PBS group, indicating that the mechanical force was insufficient for LLC to release TGF-β due to the regulation of S-HA-9, in other words, the activation of TGF-β from LLC was inhibited; and the amount of active TGF-β in the LLC+S-HA-PBA-1 group was smaller than that in the LLC+S-HA-9 group, indicating that in the form of nanoparticles, it was more beneficial to inhibit the release of active TGF-β.

The description above are only exemplary examples of the present disclosure, and is not intended to limit the present disclosure. For a person skilled in the art, the present disclosure may have various changes and variations. Any modification, equivalent substitution, improvement, etc. made within the spirit and principle of the present disclosure shall fall within the scope of protection of the present disclosure.

### INDUSTRIAL PRACTICABILITY

The sulfonated hyaluronic acid compound of the present disclosure has a stronger force to interact with an LTBP protein, so that the LTBP is prevented from binding to ECM, and consequently the mechanical force is insufficient for the activation of TGF-β, and thereby the inhibition of fibrosis at the source of signal transduction is realized, which has broad application prospects and high market value in the medical field involving the treatment of fibrosis-related diseases.

1-18. (canceled)

19. A sulfonated hyaluronic acid compound having a structural formula of 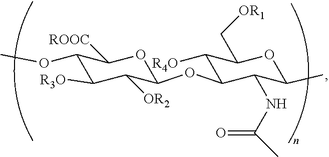
wherein R is an alkali metal cation or hydrogen; R₁, R₂, R₃ and R₄ are each independently selected from hydrogen or a sulfonate ion, and R₁, R₂, R₃ and R₄ cannot be hydrogen simultaneously; and n is an integer of 10<n<4000.

20. The sulfonated hyaluronic acid compound according to claim 19, wherein R is a sodium ion or a potassium ion or hydrogen; and R₁, R₂, R₃ and R₄ are each independently selected from hydrogen or a sulfonate ion, and R₁, R₂, R₃ and R₄ cannot be hydrogen simultaneously.

21. The sulfonated hyaluronic acid compound according to claim 19, wherein R is an alkali metal cation or hydrogen; R₁, R₂, R₃ and R₄ are a sulfonate ion; and n is an integer of 2100<n<4000.

22. A method for preparing the sulfonated hyaluronic acid compound according to claim 19, wherein the sulfonated hyaluronic acid compound is synthesized according to the following synthetic route:

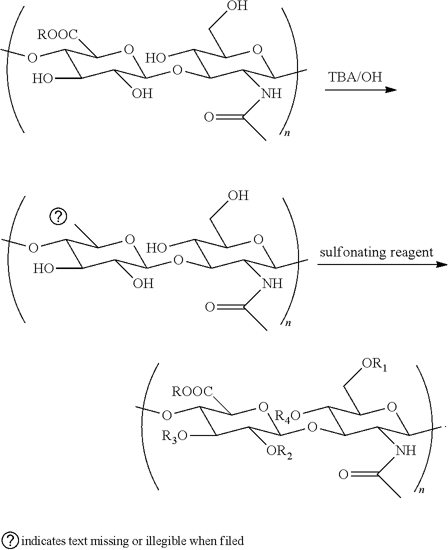

23. The method according to claim 22, wherein molecular weight of raw hyaluronic acid is 1500 kDa or less.

24. The method according to claim 23, wherein the molecular weight of the raw hyaluronic acid is any numerical value of <10 kDa or in a range of 100-200 kDa or in a range of 800 kDa-1500 kDa.

25. The method according to claim 24, wherein the molecular weight of the raw hyaluronic acid is 800 kDa-1500 kDa.

26. The method according to claim 22, wherein the sulfonating reagent is pyridine sulphur trioxide.

27. The method according to claim 22, wherein preparation of the sulfonated hyaluronic acid compound comprises the steps of dissolving the raw hyaluronic acid and then mixing with TBAOH for reaction; followed by lyophilization to form a hyaluronic acid intermediate powder; then mixing the hyaluronic acid intermediate powder with a sulfonating reagent, and adjusting the pH of reaction system to 8-9; followed by dialysis.

28. A method for inhibiting fibrosis in a subject in need thereof, comprising the step of administering to the subject the sulfonated hyaluronic acid compound according to claim 19.

29. The method according to claim 28, wherein the fibrosis comprises tissue fibrosis.

30. The method according to claim 29, wherein the tissue fibrosis comprises pulmonary fibrosis, hepatic fibrosis, cardiac fibrosis, pancreatic fibrosis or renal fibrosis.

31. A method for inhibiting activation of TGF-β in a subject in need thereof, comprising the step of administering to the subject the sulfonated hyaluronic acid compound according to claim 19.

32. A glycobiological material, comprising the sulfonated hyaluronic acid compound according to claim 19.

33. A method for inhibiting fibrosis in a subject in need thereof, comprising the step of administering to the subject the glycobiological material according to claim 32.

34. The method according to claim 33, wherein the fibrosis comprises tissue fibrosis.

35. The method according to claim 34, wherein the tissue fibrosis comprises pulmonary fibrosis, hepatic fibrosis, cardiac fibrosis, pancreatic fibrosis or renal fibrosis.

36. A method for inhibiting activation of TGF-β in a subject in need thereof, comprising the step of administering to the subject the glycobiological material according to claim 32.
